(19) United States
(12) Patent Application Publication
(10) Pub. No.: US 2024/0108233 A1
(43) Pub. Date: Apr. 4, 2024

(54) Method and Device for Collecting Physiological Data of a Wearer

(71) Applicant: Nitto Denko Corporation, Osaka (JP)

(72) Inventors: Pannawit Srisukh, Bangkok (TH); Maneerat Jiravanichkul, Bangkok (TH); Anyamanee Pornpanvattana, Bangkok (TH); Usanee Apijuntarangoon, Bangkok (TH); Visit Thaveeprungsriporn, Singapore (SG); Amornsri Khitwongwattana, Bangkok (TH)

(21) Appl. No.: 18/257,750

(22) PCT Filed: Dec. 20, 2021
(86) PCT No.: PCT/SG2021/050806

§ 371 Date: Jun. 15, 2023

(30) Foreign Application Priority Data
Dec. 18, 2020 (SG) 10202012795Q

(51) Int. Cl. A61B 5/024; A61B 5/00

(52) U.S. Cl. CPC A61B 5/02416; A61B 5/02438; A61B 5/681; A61B 2503/40

(57) ABSTRACT

A method of tuning a wearable device for collecting physiological data of a wearer, a wearable device for collecting physiological data of a wearer, a method of controlling a wearable device for collecting physiological data of a wearer, a wearable device for collecting physiological data of a wearer the method, and a computer-readable medium. The method of tuning a wearable device for collecting physiological data of a wearer comprises the steps of collecting a PPG signal using the device; determining whether or not the collected PPG signal is attributable to the wearer being human or animal; and discontinuing the tuning if the collected PPG signal is not attributable to the wearer being human or animal; wherein determining whether or not the collected PPG signal is attributable to the wearer being human or animal comprises determining information about at least one cardiac cycle based on at least one pulse in PPG signal.

### FIELD OF INVENTION

The present invention relates broadly to a method and a device for collecting physiological data of a wearer, in particular to a method of tuning a wearable device for collecting physiological data of a wearer, to a method of controlling a wearable device for collecting physiological data of a wearer, and to a wearable device for collecting physiological data of a wearer.

### BACKGROUND

Any mention and/or discussion of prior art throughout the specification should not be considered, in any way, as an admission that this prior art is well known or forms part of common general knowledge in the field.

Wearable devices which monitor biometric/physiological signals, such as a photoplethysmography, PPG, signal, and signal representative of movement, such as an accelerometer, ACC, signal, have become widely used. Such devices can assist in various forms of data tracking, such as for fitness and health related analysis, and including sleep monitoring.

For such devices, it can be important to detect if the device is not being worn, which is generally referred to herein as “wrist-off” state as compared to a “wrist-on” state. With the defect of the device not (properly) worn, the retrieval of physiological data poses a challenge for subsequent computation. Also, for such devices a reliable tuning/initialization is desired.

In addition, physiological conditions of animals may give farmers or researchers useful knowledge and insights about the animals, such as about their health, functions, and states of mind. Wearable devices can be attached on the animals' skin, such as the ear, nose, neck, head, hoof, leg, upper part of a tail, or top of a backbone. “Wrist-off” state in animal may mean “drop-off” state of the device from animal's skin.

Embodiments of the present invention seek to address one or more of the above problems.

### SUMMARY

In accordance with a first aspect of the present invention, there is provided a method of tuning a wearable device for collecting physiological data of a wearer, the method comprising the steps of:
collecting a PPG signal using the device;
determining whether or not the collected PPG signal is attributable to the wearer being human or animal; and
discontinuing the tuning if the collected PPG signal is not attributable to the wearer being human or animal;
wherein determining whether or not the collected PPG signal is attributable to the wearer being human or animal comprises determining information about at least one cardiac cycle based on at least one pulse in PPG signal.

In accordance with a second aspect of the present invention, there is provided a wearable device for collecting physiological data of a wearer, the device comprising:
a PPG sensor;
a signal collecting unit coupled to the PPG sensor for collecting a PPG signal using the PPG sensor for tuning of the device; and
a processor coupled to the signal collecting unit for determining whether or not the collected PPG signal is attributable to the wearer being human or animal;
wherein the processor is configured for discontinuing the tuning if the collected PPG signal is not attributable to the wearer being human or animal; and
wherein determining whether or not the collected PPG signal is attributable to the wearer being human or animal comprises determining information about at least one cardiac cycle based on at least one pulse in PPG signal.

In accordance with a third aspect of the present invention, there is provided a method of controlling a wearable device for collecting physiological data of a wearer, the method comprising the steps of:
collecting a PPG signal using the device;
processing the PPG signal to obtain analysis results;
determining whether or not the collected PPG signal is attributable to the device not being worn by the wearer; and
disabling display of the analysis results by the device and/or turning off a light source of the device if the collected PPG signal is attributable to the device not being worn by the wearer.

In accordance with a fourth aspect of the present invention, there is provided a wearable device for collecting physiological data of a wearer, the device comprising:
a signal collection unit for collecting a PPG signal; and
a processor coupled to the signal collection unit for processing the PPG signal to obtain analysis results;
wherein the processor is further configured to determine whether or not the collected PPG signal is attributable to the device not being worn by the wearer and to disable display of the analysis results and/or to turn off a light source of the device if the collected PPG signal is attributable to the device not being worn by the wearer.

In accordance with a fourth aspect of the present invention, there is provided a computer-readable medium comprising instructions which, when executed by a computing device, instruct the computing device to execute a method according to the first aspect and/or according to the third aspect.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a flowchart illustrating the overall algorithm flow according to an example embodiment.

FIG. 2 shows a flowchart illustrating the algorithm executed in the tuning stage according to an example embodiment.

FIG. 3 shows a diagram illustrating feature extraction for wrist-off detection according to an example embodiment.

FIG. 4 shows a comparison of measured DC level while wearing the device in resting (left side) or with high motion (right side), according to an example embodiment.

FIG. 5 (a) shows that the measured DC level is near zero when placed in dark environment, according to an example embodiment.

FIG. 5 (b) shows that the measured DC level is saturated (i.e., equal to supply voltage) when placed directly facing ambient light, according to an example embodiment.

FIG. 5 (c) shows that the measured DC level may be above zero and below saturation when placed subjected to indirect ambient light, according to an example embodiment.

FIG. 5 (d) shows that the DC level has a high variance with unstable light as a result of the device being placed (i.e., wrist-off state) with a shadow from body movement, according to an example embodiment.

FIG. 6 (a) shows the AC frequency component range of the PPG signal is typically 0.5-8 Hz, when the device according to an example embodiment is worn.

FIG. 6 (b) shows that there if there is a noise, i.e., the output signal has other frequency or frequencies (outside 0.5-8 Hz), that noise can be due to a wrist-off state with ambient light as the source such as neon lamp or display monitor, according to an example embodiment.

FIG. 7 illustrates that frequency components of a typical PPG range from 0.5-8 Hz, and that range was assumed this to be the PPG signal, while the rest of the measured range was assumed to be noise, specifically 8-50 Hz, according to an example embodiment

FIG. 8 shows a scatter plot for the features of 1) PPG DC level and 2) SNR of PPG_AC (SNR is based on rms) during wrist-on and wrist-off reference/training data, according to an example embodiment.

FIG. 9 shows a scatter plot (with enlarged inset) of variance (PPG AC) versus mean (PPG DC) for reference/training data, according to an example embodiment.

FIG. 10 (a) shows a scatter plot for the variance (PPG DC) versus mean (PPG DC), according to an example embodiment,

FIG. 10 (b) shows scatter plot for sumACSatZero versus mean (PPG DC), according to an example embodiment.

FIG. 11 shows a flow chart illustrating the wrist-off state detection according to an example embodiment.

FIG. 12 (a) illustrates determining wrist-off state based on seven consecutive time windows of PPG data, according to an example embodiment,

FIG. 12 (b) illustrates a counter threshold is set based on seven consecutive 4 s time windows of PPG data, with the wrist-off state output in each of the seven windows being counted, according to an example embodiment.

FIG. 13 illustrates the results obtained from evaluation of 21 subjects' data, yielding more than 90% accuracy of wrist-off state classification, according to an example embodiment.

FIG. 14 (a) shows example zones for high motion and low motion based on different TAT and PIM thresholds, according to an example embodiment.

FIG. 14 (b) shows example zones for high motion and low motion based on different PIM thresholds, according to an example embodiment.

FIG. 15 shows a high level flow chart illustrating a tuning and wrist-off detection method/algorithm according to an example embodiment.

FIG. 16 shows the overall schematic diagram of the device according to an example embodiment.

FIG. 17 shows a detailed diagram certain components of the device of FIG. 16, according to an example embodiment.

FIG. 18 shows a detailed diagram certain components of the device of FIG. 16, according to another example embodiment.

FIG. 19 illustrates that variance (PPG DC) and counting the sum number of saturate and the number of zero of PPG AC signal (sumACSatZero) are useful for wrist-off/off state classification, according to an example embodiment.

FIG. 20 shows a flowchart illustrating a method of tuning a wearable device for collecting physiological data of a wearer, according to an example embodiment.

FIG. 21 shows a flowchart illustrating a method of controlling a wearable device for collecting physiological data of a wearer, according to an example embodiment.

FIG. 22 shows a diagram illustrating determining a rise time and/or a fall time of a cardiac cycle included in the PPG signal, according to an example embodiment.

### DETAILED DESCRIPTION

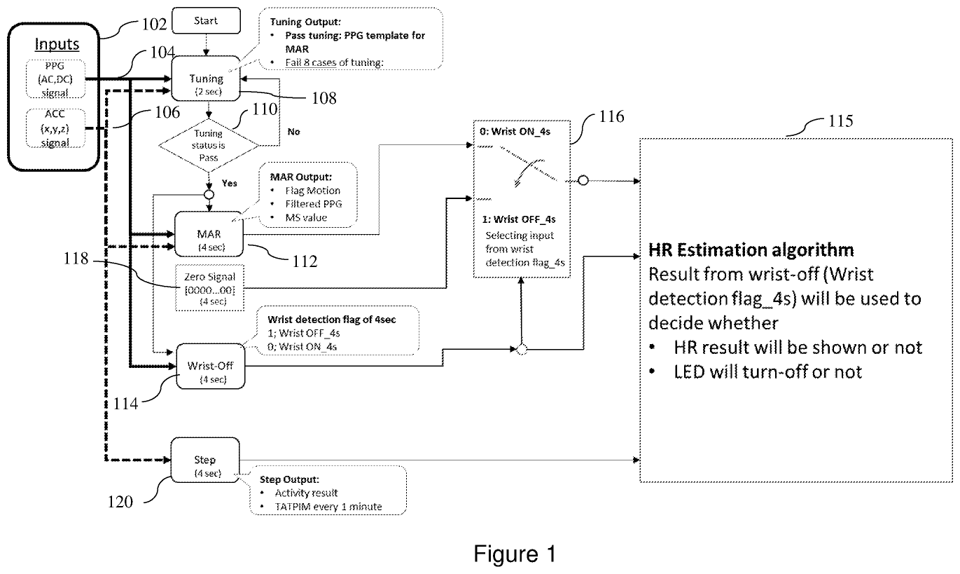

Overall algorithm flow according to an example embodiment FIG. 1 shows a flowchart illustrating the overall algorithm flow according to an example embodiment. The algorithm receives its measurement input from the wearable device 102 in the form of, generally, a photoplethysmography, PPG, signal 104 (including AC and DC components), and an accelerometer, ACC, signal 106 (including x-, y-, and z axis from respective ACC sensor components). The signal 106 may be a combination of x-y axis, y-z axis, x-z axis and/or x, y and z axis.

Both the PPG signal 104 and the ACC signal 106 are inputs to the tuning stage 108 of the algorithm. In this example embodiment, tuning requires eight conditions to be passed at decision stage 110. Specifically, in this example embodiment fail cases 1-3 are used for determining tuning failure in the form of a “wrist off”, i.e., non-worn or not properly worn device 102, and fail cases 4-8 are used for determining a general tuning failure, in an assumed “wrist on” scenario, i.e., worn device 102. It is noted that fail cases 1-3 in this example embodiment are suitable to determine that the signal is not from human/animal, as a sub-category for wrist off determination. Also, “not properly worn” here means that the user is wearing the device 102 but loose such that the PPG and/or ACC sensors do not touch the surface of the skin, or are wrongly positioned, etc.

Fail cases 1-3 in this example embodiment include:

Fail case 1: Heart rate, HR, out of range (for example, out of a range from about 30-240 bpm for humans).

Fail case 2. Light emitting device, LED, driving current for the PPG sensor is maximum (for example, ≥20 mA) but no pulses with information about a cardiac cycle within a certain range are identified from the PPG signal (for example, at least 3 pulses with rise time, 10%≤RT≤50%, where RT means the rise time percentage in one cardiac cycle).

Fail case 3. PPG signal amplitude <28%, for example, of an acceptable amplitude (acceptable amplitude may be set in the range from about 20-70 mV)

If any one of the above 3 fail cases is detected, it is considered as wrist off status in this example embodiment. Responsive to the determination of the wrist off status, the LED sensor of the device 102 is turned off until the device 102 detects movement by checking the ACC signal 106 against time-above threshold, TAT, thresholds and/or proportional integration mode, PIM, thresholds. FIG. 14 (a) shows example zones for high motion and low motion based on different TAT and PIM thresholds, according to an example embodiment. High motion thresholds can be 100 for TAT and 12 for PIM, and low motion thresholds can be 5 for TAT and 12 for PIM. Upon determining high motion movement based on the TAT/PIM threshold, the LED is turned back on for the next 5 minutes and tuning in the tuning stage 108 is re-started. FIG. 14 (b) shows another example, with zones for high motion and low motion based on different PIM thresholds (only), where the PIM threshold can be about 12-85 in some example embodiments.

Fail cases 4-8 in this example embodiment include:

Fail case 4: Average of DC level >83%, for example, of supply voltage.

Fail case 5: LED driving current is maximum but the PPG amplitude is not acceptable, as judged by the acceptable amplitude.

Fail case 6: LED driving current is minimum (for example, threshold is 1 mA) and the PPG amplitude >50% of supply voltage.

Fail case 7: Processing time >24 s, or more generally a threshold between about 8 sec-4 min in example embodiments, at the same LED driving current, the timer being reset at 0 when driving current level is changed during the tuning process.

Fail case 8: Motion detected from ACC is continuously high for a certain period e.g., 10 seconds. It is noted that any other predefined period may also be set and considered.

If any one of the above fail cases is detected, tuning terminates and the tuning will be reactivated in the next 5 minutes, in one example embodiment.

If the tuning decision stage 108 is a pass, the motion artifact removal, MAR, stage 112 algorithm will be processed. Outputs of the MAR stage 112 are Flag motion, Filtered PPG, and motion strength, MS, value.

Also, if the tuning decision stage 108 is a pass, the wrist-off stage 114 algorithm will be processed. An example embodiment of the wrist-off stage 114 algorithm will be described in detail below. The result from wrist-off (Wrist detection flag_4 s) will be used to decide whether further processing results of further algorithms, indicated as stage 115, such as HR, will be shown/displayed or not even though tuning is passed, and whether the sensor LED will be turned off or not. In this embodiment, the output from the wrist-off stage 114 is used to control a switch 116 to either feed the output from the MAR stage 112 to the further algorithm stage 115, or a zero signal from a zero signal generator stage 118. It is noted that depending on the purpose of the further algorithm or wavelength of the light, the signal may not be required to enter the MAR stage after passing the tuning stage in different example embodiments.

Throughout the execution of the algorithm, the Step stage 120 outputs an activity result, and TAT/PIM measured, every 1 minute. Briefly, Step stage 120 is to count steps to monitor user's activity. An example step count algorithm can be as described in WO/2015/183193.

Details of the Tuning Stage Algorithm According to an Example Embodiment

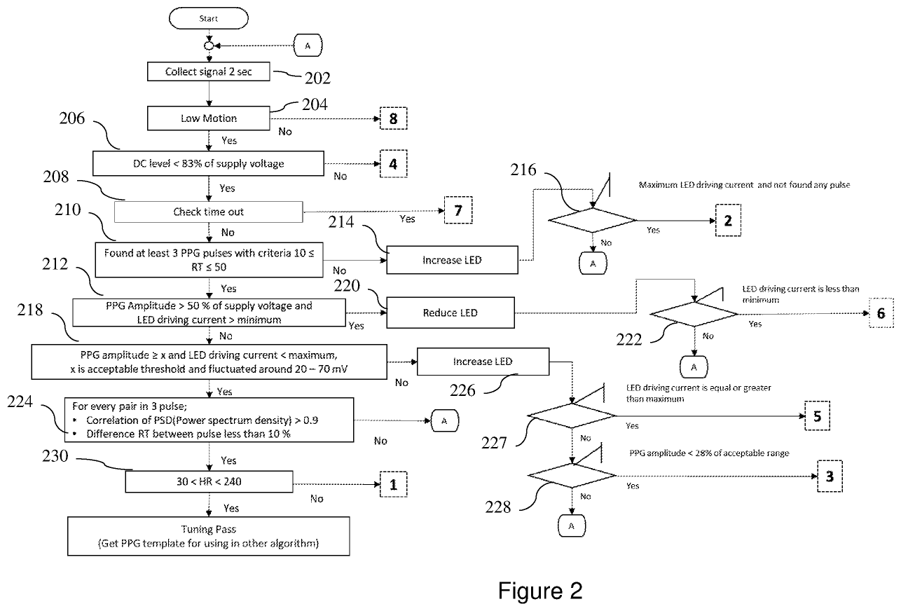

FIG. 2 shows a flowchart illustrating the algorithm executed in the tuning stage according to an example embodiment. After the device is turned on, the system will start tuning. There may be a task schedule, which can be up to 5 mins in one example embodiment. For example, if the device is turned on at 10:02 AM, then tuning starts at 10:05 for a device configured to start the tuning algorithm at X:05, X:10, X:15 etc. Generally, the tuning process includes adjusting the sensor LED level and verifying detected signals.

Specifically, in this example embodiment the Collect stage 202 collects PPG and ACC signals signal for 2 sec, and Low Motion stage 204 checks the fail case 8 criteria, e.g., if the strength of intensity and/or the frequency of the ACC signal representative of motion equal or less than e.g., 4 km/hr of walking or e.g., 6.5 km/hr of running Otherwise, the fail case 8 criteria for high motion is considered to have been triggered, i.e., “No” output from Low Motion stage 204.

Next, the nonpulsatile component, DC, level stage 206 checks the fail case 4 criteria, e.g., if the DC level of the PPG signal is less than a threshold portion of the sensor LED supply voltage, tuning proceeds. The threshold portion can be around 83% of the sensor LED supply voltage in this example embodiment. On the other hand, if the DC level is equal to or greater than the threshold portion, the fail case 4 criteria for high DC level is considered to have been triggered, i.e., “No” output from DC level stage 206.

Next, the Time usage stage 208 checks the fail case 7 criteria. In this example embodiment, if the time usage for the tuning is greater than a threshold, for example, 24 sec, or generally within about 8 sec to 4 min, tuning proceeds to PPG pulse stage 210. On the other hand, if the time usage is outside that range, the fail case 7 time usage criteria is considered to have been triggered, i.e., “No” output from Time usage stage 208.

Next, the PPG pulse stage 210 in this example embodiment checks whether at least 3 PPG pulses in the pulsatile component, AC, component of the PPG signal with criteria rise time, within 10%≤RT≤50% are found, as a non-limiting example of the information about at least one cardiac cycle based on at least one pulse in the PPG signal. If “Yes”, the tuning proceeds to the PPG amplitude stage 212. If “No”, the sensor LED driving current is increased, indicated as stage 214, and a determination is made whether the sensor LED driving current is at the applicable maximum, for example, 20 mA, indicated at stage 216. If the sensor LED is at maximum driving current (or LED intensity) and at least 3 PPG pulses were not found, then fail case 2 is considered triggered. Otherwise, the tuning loops back as indicated by “A” in FIG. 2.

The 1^{st} PPG amplitude stage 212 checks whether the PPG AC amplitude is more than a threshold portion of the sensor LED supply voltage, and whether the sensor LED driving current is more than a minimum value, for example, 1 mA. The threshold portion can be 50% in this example embodiment. If the output from the 1^{st} PPG amplitude stage 212 is “No”, the tuning proceeds to the 2^{nd} PPG amplitude stage 218. If the output from the 1^{st} PPG amplitude stage 212 is “Yes”, the sensor LED driving current is decreased, indicated as stage 220, and a determination is made whether the sensor LED driving current is equal to (or below) the applicable minimum, indicated at stage 222. If the sensor LED is at the minimum driving current (or LED intensity) at stage 222, then fail case 6 is considered triggered. Otherwise, the tuning loops back as indicated by “A” in FIG. 2.

The 2^{nd} PPG amplitude stage 218 checks whether the PPG AC amplitude is equal to or more than an acceptable threshold and whether the LED driving current is less than the applicable maximum. The acceptable threshold can be around 20-70 mV in this example embodiment. If the output from the 2^{nd} PPG amplitude stage 218 is “Yes”, the tuning proceeds to the PPG pulse pair stage 224. If the output from the 2^{nd} PPG amplitude stage 218 is “No”, the sensor LED driving current is increased, indicated as stage 226, and a determination is made whether the sensor LED driving current is equal to (or above) the applicable maximum, indicated at stage 227.

If the sensor LED is at the maximum driving current (or LED intensity) at stage 227, then fail case 5 is considered triggered. Otherwise, a determination is made whether the PPG amplitude is smaller than a threshold portion of the acceptable amplitude, indicated at stage 228. The threshold portion can be 28% in this example embodiment. If “Yes”, fail case 3 is considered triggered. If “No”, the tuning loops back as indicated by “A” in FIG. 2.

In the PPG pulse pair stage 224, for every pair in (at least) 3 pulses it is checked whether the correlation of PSD (power spectrum density) is more than a correlation threshold, and whether a difference in RT is less than a given RT difference threshold, as a non-limiting example of the information about at least one cardiac cycle based on at least one pulse in the PPG signal. The correlation threshold can be 90% and the RT difference threshold can be 10% in this example embodiment. Correlation of PSD can be correlation of pulse shape in this example embodiment. If the output from the PPG pulse stage 224 is “Yes”, the tuning proceeds to HR stage 230. If the output from the PPG pulse stage 224 is “No”, the tuning loops back as indicated by “A” in FIG. 2.

It is noted that another condition can be added in PPG pulse stage 224 to check the difference of HR between every pair in 3 PPG pulses and determine whether any of the differences is less than about 20-35% of normal resting HR. In one example embodiment, the absolute difference in HR between 2 pulses being less than 20 bpm is used, however, more generally less than about 20-35% may be used in example embodiments, which equates to about 12-35 bpm.

The HR stage 230 checks whether the heart rate, HR, is within the range of 30<HR<240. If the output from HR stage 230 is “No”, fail case 1 is considered triggered. Otherwise, tuning is considered a pass, and a PPG template is constructed for use in other parts of the overall algorithm.

It is noted that in this example embodiments, the algorithm can be applicable to both human and animals (cow, sheep, etc). In an example embodiment specific to human (only), the heart rate, HR, range e.g., can be 30<HR<120.

Similarly, in an example embodiment specific to human (only), the rise time range (compare PPG pulse stage 210) can e.g., be 10%≤RT≤40%, as a non-limiting example of the information about at least one cardiac cycle based on at least one pulse in the PPG signal.

For each of fail cases 1-3, the device will turn off the sensor LED until motion is detected by checking TAT and/or PIM threshold, as described above with reference to FIG. 1.

It is noted that in different example embodiment, the number, type, and or order/sequence of decision making steps and associated processing for determining respective fail cases may be changed compared to the flowchart shown in FIG. 2.

Details of the Wrist-Off Stage Algorithm According to an Example Embodiment

The algorithm executed in the wrist-off stage (compare numeral 114 in FIG. 1) according to an example embodiment identifies a wrist-off status if the user is not wearing the device (or not properly wearing the device), and a wrist-on status if user is wearing the device.

While user wears the device (wrist-on state), the wear condition may be resting or moving, noting that resting can include sleeping. Moving includes irregular movements such as, but not limited to, coding (i.e., typing on a computer/keyboard), lecture, discussion etc., and regular movements such as, but not limited to, running, walking, cycling etc.

During the wrist-off state, the user is not wearing the device, and there can be stable or moving conditions as follows, by way of example, not limitation:

Stable condition can include 1) placed on table with bright environment (Neon light, Display monitor, sunlight, etc), 2) placed on table with shadow of body movement, 3) placed in dark environment (e.g., in dark room or bag, etc).

Moving condition can include 1) placed in dark environment with movement (keep the device in a bag while walking with the bag etc), 2) placed in bright environment with movement (e.g., holding in hand while walking, etc).

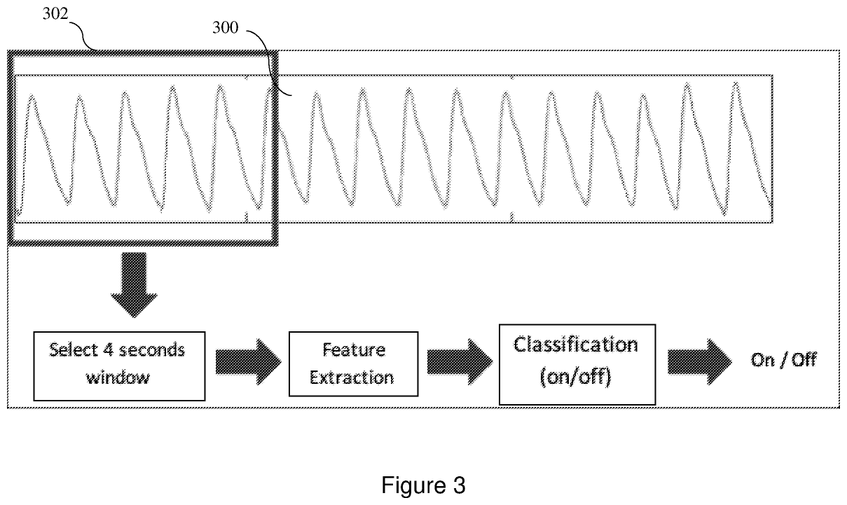

FIG. 3 shows a diagram illustrating feature extraction for wrist-off detection according to a non-limiting example embodiment.

The PPG signal 300 is separated into 4 seconds windows e.g., 302, each with 332 data points, indicated as step 303, and the data is mapped into new dimension, i.e., into the extracted features (5 features in one example embodiment for further calculation), indicated as step 304. It is noted that each window e.g., 302 period may be of any predefined duration, noting that, preferably, 2 beats/pulses e.g., 305 are included in each window. Generally, accuracy may change due to the window size, with larger window size typically providing better accuracy, while smaller window size typically provides less processing amount.

Generally, feature data=(feat1, feat2, feat3, . . . ) is extracted and a classification into wrist-on or wrist-off is made, indicated as step 306, and a corresponding output signal is being generated, indicated at step 308.

Listed below are features calculated for each window e.g., 302 according to a non-limiting example embodiment:
1) Average DC level of PPG signal
2) Signal to noise ratio of AC component of PPG signal in frequency domain
3) Variance in DC level of PPG signal in time domain
4) Variance in AC component of PPG signal in time domain
5) Sum of number of saturate points and zero points of AC component in PPG signal in time domain

In one example embodiment, main features are 1) Average DC level and 2) Signal to noise ratio of AC component in frequency domain, whereas 3) Variance in DC level, 4) Variance in AC component, and 5) Sum of number of saturate points and zero points of AC components are optional features to be used for specific cases. Details of the features will now be described.

1) PPG DC Level

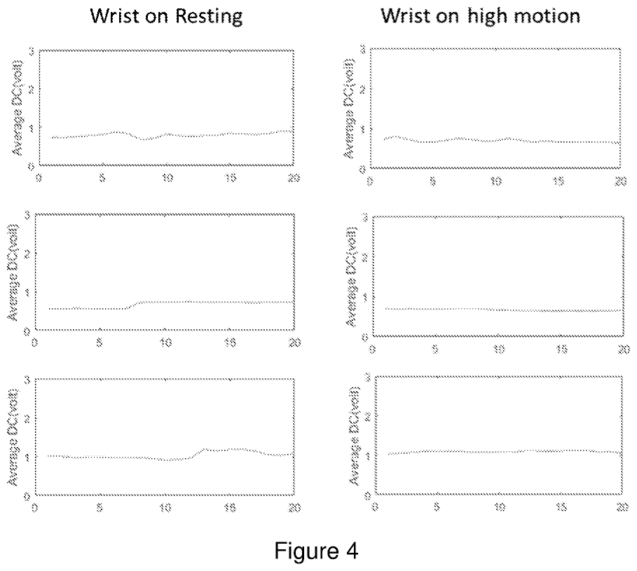

FIG. 4 shows a comparison of measured DC level while wearing the device in resting (left side) or with high motion (right side). As can been seen from the results in FIG. 4, body movement does not affect the DC level discernably.

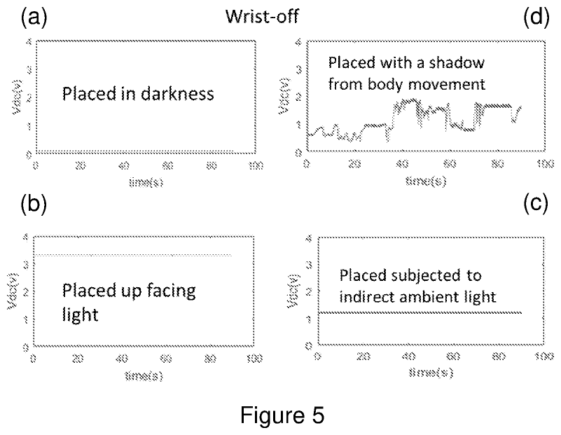

FIG. 5 (a) shows that the measured DC level is near zero when placed in dark environment. FIG. 5 (b) shows that the measured DC level is saturated (i.e., equal to supply voltage) when placed directly facing ambient light. This may be used to distinguish those scenarios from a wrist-on state.

Accordingly, the DC level of the PPG signal was found to be a useful parameter to identify wrist-off state when the device is placed in dark environment, or when placed directly facing ambient light, according to an example embodiment.

However, FIG. 5 (c) shows that the measured DC level may be above zero and below saturation when placed subjected to indirect ambient light. This may not be useful to distinguish from a wrist-on state.

FIG. 5 (d) shows that the DC level has a high variance with unstable light as a result of the device being placed (i.e., wrist-off state) with a shadow from body movement. However, it was found that the variance of the DC level of wrist-off state in 5 (d) is possibly similar to the wrist-on state, where reflected light may vary depending on the condition of user's anatomy (skin, tissue, or bone) and the DC level may be 0.1-2.2 Vdc.

2. PPG_AC

The signal to noise ratio, SNR, of the AC component of the PPG signal in frequency domain was found to be efficient to identify noise by ambient light in the wrist-off stage.

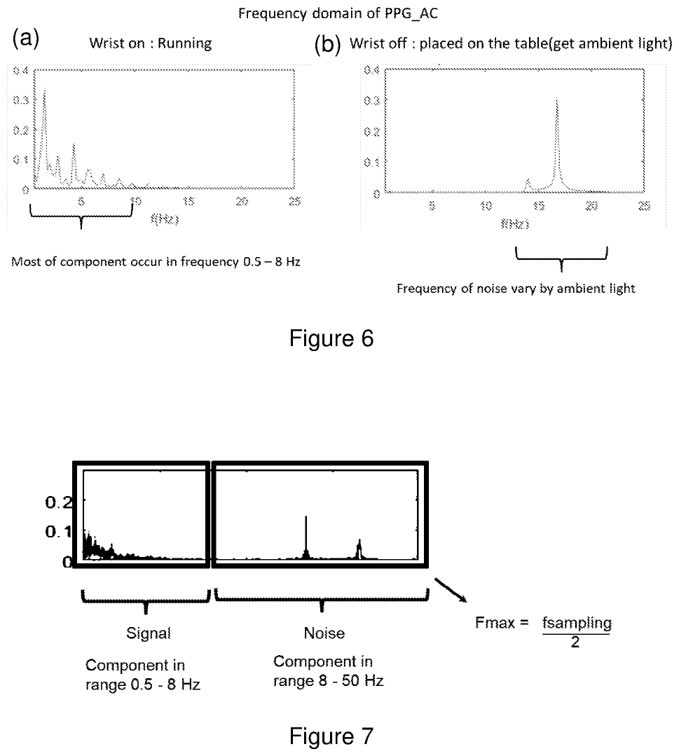

When worn, the AC frequency component range of the PPG signal is typically 0.5-8 Hz, as shown in FIG. 6 (a). It was found that if there is a noise, i.e., the output signal has other frequency or frequencies (outside 0.5-8 Hz), as shown in FIG. 6 (b), that noise can be due to a wrist-off state with ambient light as the source such as neon lamp or display monitor.

In an example embodiment SNR is used to check the characteristics of the signal transmitter and receiver, i.e., the sensor LED and photodetector, respectively, by comparing the level (or power) of the desired signal with the level (or power) of noise. It is noted that compared to analyzing the signal in time domain (Amplitude vs time) to calculate signal to noise ratio, it was found that analyzing the signal in frequency domain (Amplitude vs frequency) is preferred because the signal in the frequency domain shows the dominant frequency, i.e., signals can be categorized according to their frequencies.

In an example embodiment, the root mean square, rms, of the AC frequency domain amplitude in the PPG signal range and the rms of amplitude in the noise range are compared. As mentioned above, according to frequency components of a typical PPG range from 0.5-8 Hz, that range was assumed this to be the PPG signal, while the rest of the measured range was assumed to be noise, specifically 8-50 Hz in an example embodiment, and as shown in FIG. 7.

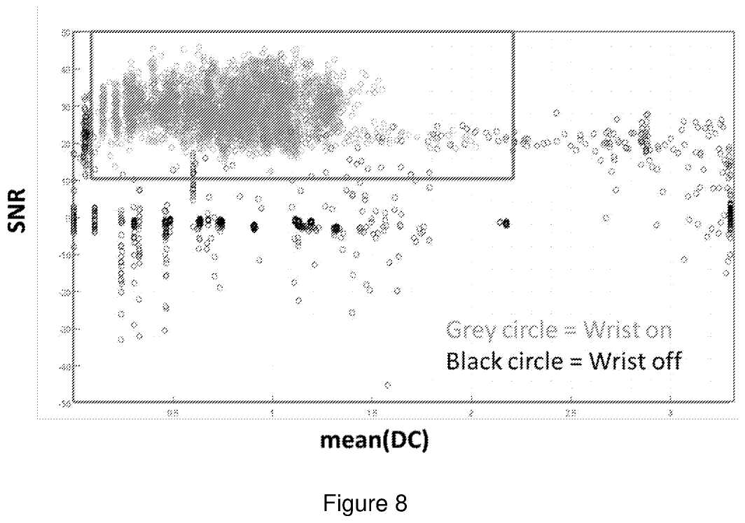

To identify wrist-on/off state according to an example embodiment, a threshold can be predetermined for each feature. FIG. 8 shows a scatter plot for the features of 1) PPG DC level and 2) SNR of PPG_AC (SNR is based on rms as described above, in one example embodiment) during wrist-on and wrist-off reference/training data. The rectangular 800 represents the cluster for a condition of wrist-on state according to an example embodiment. The plot in FIG. 8 shows wrist-on and wrist-off data are well separated. Thus, thresholds for a classification model according to example embodiments can be set.

In one example embodiment, wrist-on conditions may be set to: Mean (PPG DC level)=0.001-2.5 V, and SNR PPG_AC >5-10 dB.

Although the two main features of mean (PPG DC level) and SNR PPG_AC are able to classify the data with high accuracy according to an example embodiment, there are some cases where these two main features may not be sufficient/accurate enough to identify wrist-on/off state.

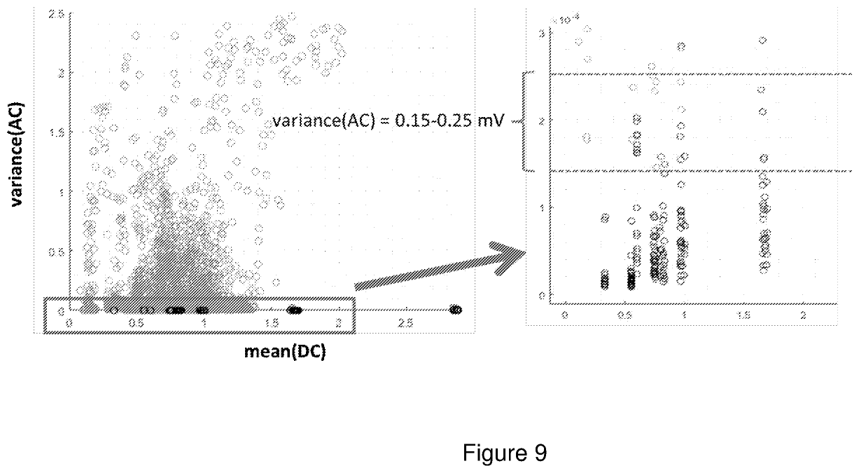

One case can be the wrist-off state when the device is placed in direct sunlight, without any electronic light. On the other hand, it was found that PPG_AC signal has an extremely low amplitude under direct sunlight condition. Thus, this case can be classified based on a threshold for the variance (PPG_AC), for example, lower than 0.15-0.25 mV in one example embodiment. FIG. 9 shows a scatter plot (with enlarged inset) of variance (PPG AC) versus mean (PPG DC) for reference/training data, where darker line circles indicate the wrist-off state when the device is placed in direct sunlight, from which the threshold can be set according to an example embodiment.

Another case can be the wrist-off state with holding the device in hand, where it was found that there is high variance in both DC and AC PPG signal components up to the limit of the supply voltage, and including the low (zero) boundary equivalent to zero supply voltage. Reaching (or exceeding) the limit of supply voltage results in signal saturation at high (saturated), and going to zero at the low (zero) boundary of supply voltage, and the AC signal was found to show both saturate and zero. Thus, variance (PPG DC) and counting the sum number of saturate and the number of zero of PPG AC signal (sumACSatZero) are useful in an example embodiment. Specifically, it was found by the inventors that number of saturate and the number of zero of PPG AC signal (sumACSatZero) are very distinctive between wrist-off and wrist-on states, as illustrated in FIG. 19.

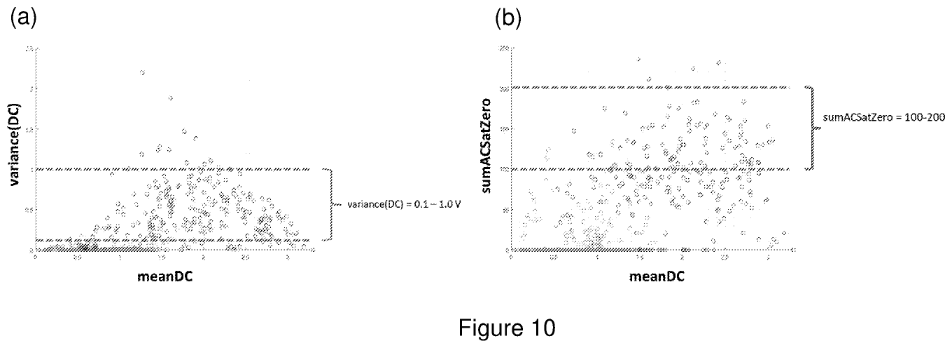

FIGS. 10 (a) and (b) show scatter plots for the variance (PPG DC) versus mean (PPG DC) and sumACSatZero versus mean (PPG DC), respectively, with wrist-off holding in hand reference/training data indicated as lighter line circles, from which the threshold can be set according to an example embodiment, for example, variance (DC)≥0.1-1.0 and/or sumACSatZero ≥100-200.

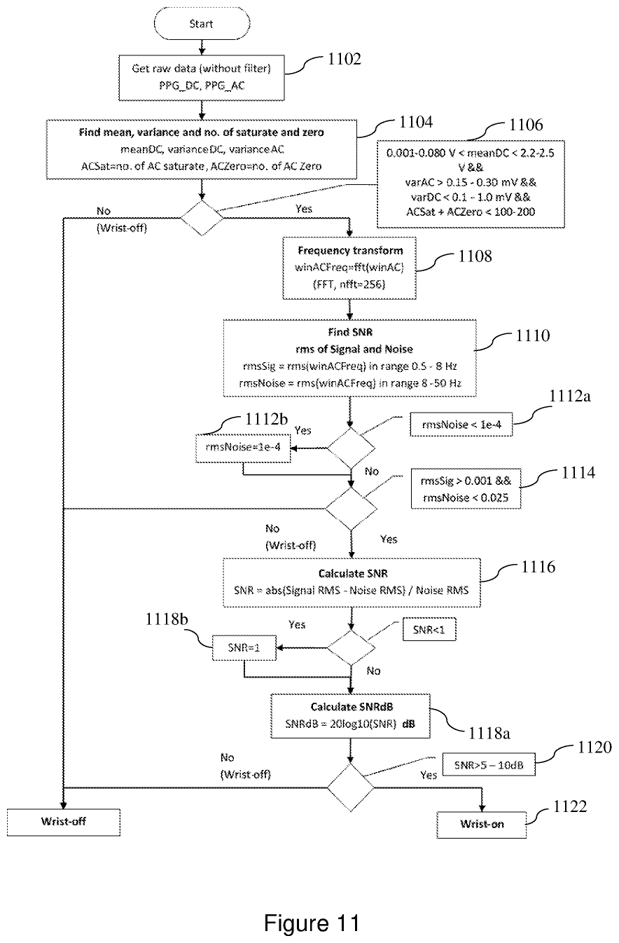

FIG. 11 shows a flow chart illustrating the wrist-off state detection according to an example embodiment.

At step 1102, raw data of PPG DC and AC is collected with 4 seconds window, i.e., without filtering such as finite impulse filtering.

At step 1104, mean PPG DC, Variance PPG DC, Variance PPG AC, Sum number of saturate points and zero points of PPC AC are calculated.

At step 1106, the calculated mean PPG DC, Variance PPG DC, Variance PPG AC, Sum number of saturate points and zero points of PPC AC are compared with respective thresholds for the wrist-on state, and if not all conditions are met, i.e., output from step 1106 is “No”, the state is judged as wrist-off. Values for the thresholds are shown according to an example embodiment.

At step 1108, if the output from step 1106 was “Yes”, the PPG AC signal is transformed to frequency domain.

At step 1110, the rms values in the signal range and in the noise range, respectively are first calculated in the frequency domain, as part of the later calculation of the SNR (see step 1116 below). Values for the ranges are shown according to an example embodiment.

At steps 1112a and 1112b, the rms for the noise signal is bound to a minimum value, to avoid calculation errors in subsequent calculation. A value for the minimum is shown according to an example embodiment.

At step 1114, rms of the PPG AC signal and rms of the PPG AC noise are compared with respective thresholds for the wrist-on state, and either of both conditions are not met, the state is judged as wrist-off. Values for the thresholds are shown according to an example embodiment.

At step 1116, the SNR is calculated based on the ratio of the rms of the PPG AC signal and the rms of the PPG AC noise.

At step 1118a and 1118b, the calculated SNR is bound to a minimum value, to avoid calculation errors in subsequent calculation. A value for the minimum is shown according to an example embodiment.

At step 1120, the SNR in dB is calculated.

At step 1122, the SNR in dB is compared with a threshold. If the result is “No”, the state is judged as wrist-off. If the result is “Yes”, the state is judged as wrist-on. A value for the threshold is shown according to an example embodiment.

In an example embodiment, a counter for the wrist-off state determination is implemented. This can help to ensure disabling display of PPG/ACC data analysis algorithm results, such as HR result and turn off the sensor LED rather than show inappropriate results, only during “true” wrist-off state, In other words, the accuracy can be improved in an example embodiment by using a predetermined number of output results of wrist-off state to be reached before disabling display of PPG/ACC data analysis algorithm results, rather than based on each individual output result.

It is noted that in different example embodiment, the number, type, and or order/sequence of decision making steps and associated processing for classifying wrist-on/off states may be changed compared to the flowchart shown in FIG. 11.

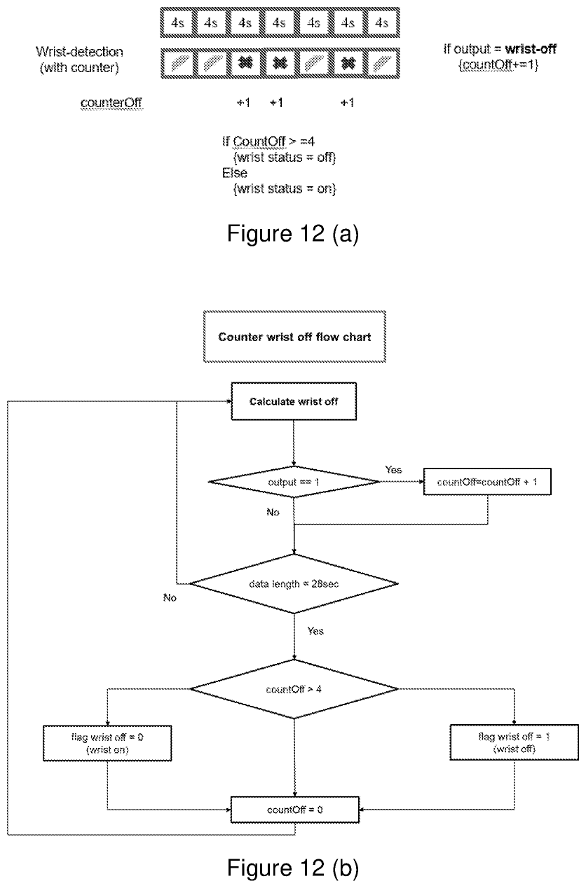

In an example embodiment, a counter threshold is set based on seven consecutive 4 s time windows, with the wrist-off state output in each of the seven windows being counted, as illustrated in FIGS. 12 (a) and 12 (b). If wrist-off states are shown for more than half of consecutive 4 s time windows, overall wrist status is determined as wrist-off and display of PPG/ACC data analysis algorithm results is disabled or displayed as “NA”. In this example embodiment, if wrist-off states were shown greater than or equal to 4 times, overall wrist state is judged as wrist-off state. If wrist-off states were shown less than 4 times, overall wrist state is judged as wrist-on state. This process is continued for the next 7 windows if the overall wrist state is judged wrist-on state, etc.

Results of Wrist-on/Off Classification According to an Example Embodiment

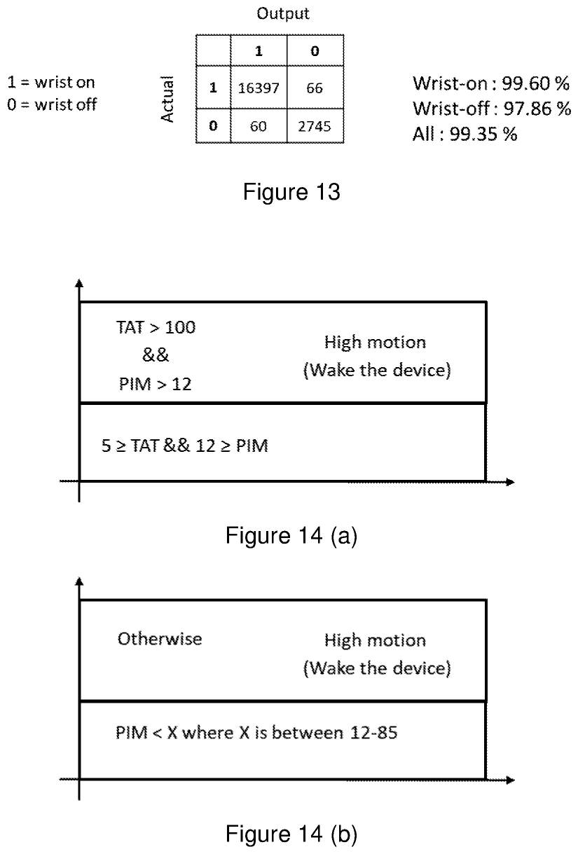

FIG. 13 illustrates the results obtained from evaluation of 21 subjects' data, yielding more than 90% accuracy of wrist-off state classification.

The wrist-on conditions used in the evaluation were:
Mean (PPG DC) 0.001-0.080<mean (PPG DC)<2.2-2.5 V;
SNR (PPG AC)>5-10 dB;
Variance (PPG AC)>0.15-0.30 mV;
Variance (PPG DC)<0.1-1.0 mV;
No. of PPG AC saturate+no. of PPG AC zero <100-200 points.

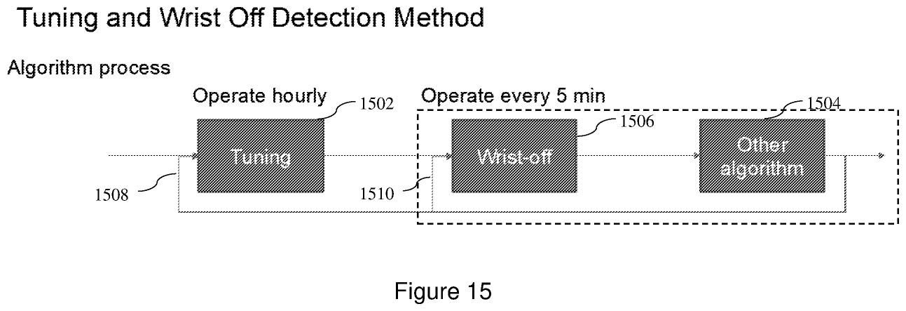

FIG. 15 shows a high level flow chart illustrating a tuning and wrist-off detection method/algorithm according to an example embodiment. The tuning stage 1502 is operated hourly in this example embodiment, and upon passing the tuning stage 1502, other algorithms are executed as indicated in stage 1504, with the wrist-off detection stage 1506 operated every 5 minutes in this example embodiment. The loop arrows 1508, 1510 indicate the periodic operation of the tuning stage 1502 and the wrist-off detection stage 1506, respectively. It is noted that the timing may be different in various example embodiments and is not limited to the example timings shown in FIG. 15.

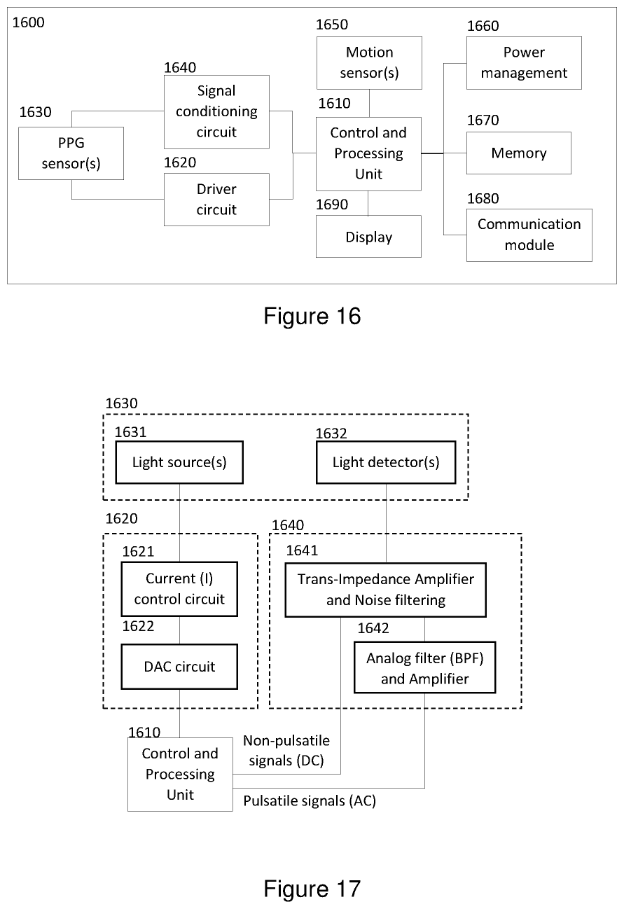

In the following, a general description of the wearable device 102 (FIG. 1) according to example embodiments will be provided. FIG. 16 shows the overall schematic diagram of the device 102. The PPG sensor(s) 1630 are connected to a signal conditioning circuit 1640 and a driver circuit 1620, which in turn are connected to the Control and Processing Unit, CPU, 1610 which contains the tuning and wrist-off detection algorithms according to example embodiments and the CPU 1610 is also connected to the motion sensor(s) 1650, a display 1690, power management unit 1660, memory 1670, and communication module 1680.

FIG. 17 is the detailed diagram of the components 1610, 1620, 1630, 1640, showing the driving current control component 1621 (for adjusting the LEDs' intensity) as well as the digital to analog converter (DAC) circuit 1622, which converts the digital value commanded by MCU to be analog value for current control circuit 1621. PPG sensor(s) 1630 include light sources(s) 1631 and light detector(s) 1632. Light sources(s) 1631 are connected to current control circuit 1621 and DAC circuit 1622 to control intensity. Light detector(s) 1632 detect light, and the detected signals are processed with Trans-Impedance Amplifier and Noise filtering components 1641 to obtain Non-pulsatile signals (DC component), and further processed in Analog filter (BPF) and Amplifier 1642 to obtain Pulsatile signals (AC component). AC component and DC component of signals are sent to control and processing unit 1610 for further processes and computations.

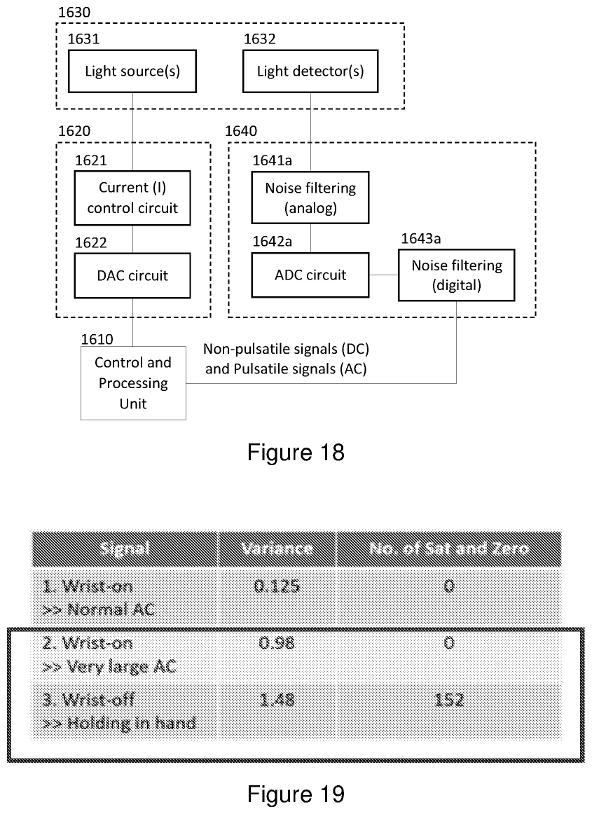

In an alternative embodiment shown in FIG. 18, the signal conditioning circuit 1640 can comprise a Noise filtering (analog) component 1641a, an ADC circuit 1642a and a noise filtering (digital) component 1643a instead which process the detected signals to obtain non-pulsatile (DC) and pulsatile (AC) signals in digital form and send to control and processing unit 1610 for further processes and computations.

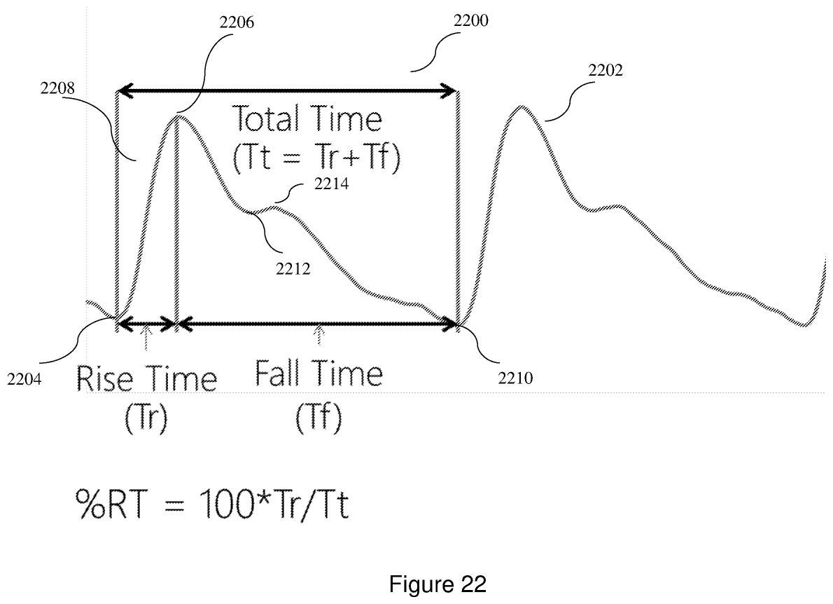

FIG. 22 shows a diagram illustrating determining a rise time and/or a fall time of a cardiac cycle 2200 included in the PPG signal 2202, according to an example embodiment. The CPU 1610 (FIG. 16) is configured to detect a first valley position 2204 and a systolic peak position 2206 and a second valley position 2210 of the cardiac cycle 2200 (and hence the PPG pulse 2208). It should be appreciated that the filtered PPG signal 2202 also includes a dicrotic notch 2212 and a diastolic peak 2214, which may also be used in example embodiments for determining whether or not the collected PPG signal is attributable to the wearer being human or animal.

The CPU 1610 (FIG. 16) is configured to calculate a rise time based on the first valley position 2204 and the systolic peak 2206 of the cardiac cycle 2200. Mathematically, referring to FIG. 22, the rise time, RT, for the cardiac cycle 2200 is RT=100×RT (Rising Time)/Total time of the PPG pulse 2208. A typical range for RT in humans is about 10-40%, and in animals about 10-50%. Alternatively, or additionally, the CPU determines a fall time of cardiac cycle 2200. Mathematically, the fall time, FT, is FT=total time of the PPG pulse 2208—RT. A typical range for FT in humans is about 60-90%, and in animals about 50-90%.

That is, in the tuning algorithm according to an example embodiment, determining whether or not the collected PPG signal is attributable to the wearer being human or animal can comprise determining information about at least one cardiac cycle 2200 based on the PPG pulse 2208, and more specifically can comprise determining one or more of a group consisting of a rise time, a fall time, time information relating to the dicrotic notch 2212, time information relating to the diastolic peak 2214, and information about the shape of the PPG pulse 2208 or the shape(s) of one or more portions of the PPG pulse 2208. Similarly, determining whether a difference in the information about the at least one cardiac cycle is less than a threshold can comprise determining the difference in one or more of a group consisting of a rise time, a fall time, time information relating to the dicrotic notch 2212, time information relating to the diastolic peak 2214, and information about the shape of the PPG pulse 2208 or the shape(s) of one or more portions of the PPG pulse 2208.

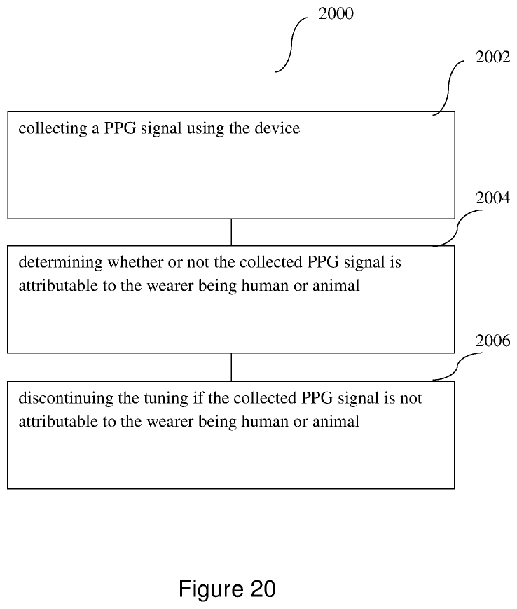

FIG. 20 shows a flowchart 2000 illustrating a method of tuning a wearable device for collecting physiological data of a wearer, according to an example embodiment. At step 2002, a PPG signal is collected using the device. At step 2004, it is determined whether or not the collected PPG signal is attributable to the wearer being human or animal; and at step 2006, the tuning is discontinued if the collected PPG signal is not attributable to the wearer being human or animal; wherein determining whether or not the collected PPG signal is attributable to the wearer being human or animal comprises determining information about at least one cardiac cycle based on at least one pulse in PPG signal.

The information about the at least one cardiac cycle may comprise one or more of a group consisting of a rise time, a fall time, time information relating to the dicrotic notch, time information relating to the diastolic peak, and information about the shape of the at least one pulse in the PPG signal or the shape(s) of one or more portions of the at least one pulse in the PPG signal.

The method may comprise, when it is determined that the information about the at least one cardiac cycle is not within the predetermined range, increasing an intensity of a light source for the PPG measurements. The method may comprise determining whether a driving current of the light source has reached a maximum, and determining a fail of tuning if yes. The method may comprise repeating collecting a PPG signal using the device if the driving current of the light source has not reached the maximum.

The method may comprise determining whether an amplitude of the PPG signal is greater than a first threshold and whether the driving current of the light source is above a minimum, and reducing the intensity of the light source if both conditions are fulfilled. The method may comprise determining whether the driving current of the light source has reached the minimum, and determining a fail of tuning if yes. The method may comprise repeating collecting a PPG signal using the device if the driving current of the light source has not reached the minimum.

The method may comprise determining whether an amplitude of the PPG signal is equal or greater than a second threshold and whether the driving current of the light source is below the maximum, and increasing the intensity of the light source if both conditions are fulfilled. The method may comprise determining whether the driving current of the light source is equal to or greater than the maximum and determining whether the PPG amplitude is smaller than a third threshold, and determining a fail of tuning if at least one of the conditions is fulfilled. The method may comprise repeating collecting a PPG signal using the device if both conditions are not fulfilled.

The method may comprise determining whether in a plurality of pairs of pulses in the PPG signal a correlation of a power spectrum density is above a fourth threshold and determining whether a difference in the information about the at least one cardiac cycle is less than a fifth threshold, and repeating collecting a PPG signal using the device if at least one of the conditions is not fulfilled.

The method may comprise determining whether in a plurality of pairs of pulses in the PPG signal a correlation of a power spectrum density is above a fourth threshold, whether a difference in the information about the at least one cardiac cycle is less than a fifth threshold, and whether the difference in HR is less than sixth threshold relative to a normal resting heart rate, and repeating collecting a PPG signal using the device if at least one of the three conditions is not fulfilled.

The method may comprise determining whether HR is in a normal range for a human and/or animal.

In one embodiment, a wearable device for collecting physiological data of a wearer is provided, the device comprising a PPG sensor; a signal collecting unit coupled to the PPG sensor for collecting a PPG signal using the PPG sensor for tuning of the device; and a processor coupled to the signal collecting unit for determining whether or not the collected PPG signal is attributable to the wearer being human or animal; wherein the processor is configured to discontinue the tuning if the collected PPG signal is not attributable to the wearer being human or animal; and wherein determining whether or not the collected PPG signal is attributable to the wearer being human or animal comprises determining information about at least one cardiac cycle based on at least one pulse in PPG signal.

The information about the at least one cardiac cycle may comprise one or more of a group consisting of a rise time, a fall time, time information relating to the dicrotic notch, time information relating to the diastolic peak, and information about the shape of the at least one pulse in the PPG signal or the shape(s) of one or more portions of the at least one pulse in the PPG signal.

The processor may be configured, when it is determined that the information about the at least one cardiac cycle of pulses in the PPG signal is not within the predetermined range, increasing an intensity of a light source of the PPG sensor for the PPG measurements. The processor may be configured to determine whether a driving current of the light source has reached a maximum, and to determine a fail of tuning if yes. The signal collecting unit may be configured to repeat collecting a PPG signal if the driving current of the light source has not reached the maximum.

The processor may be configured to determine whether an amplitude of the PPG signal is greater than a first threshold and whether the driving current of the light source is above a minimum, and to reduce the intensity of the light source if both conditions are fulfilled. The processor may be configured to determine whether the driving current of the light source has reached the minimum, and to determine a fail of tuning if yes. The signal collecting unit may be configured to repeat collecting a PPG signal if the driving current of the light source has not reached the minimum.

The processor may be configured to determine whether an amplitude of the PPG signal is equal or greater than a second threshold and whether the driving current of the light source is below the maximum, and to increase the intensity of the light source if both conditions are fulfilled. The processor may be configured to determine whether the driving current of the light source is equal to or greater than the maximum and determining whether the PPG amplitude is smaller than a third threshold, and to determine a fail of tuning if at least one of the conditions is fulfilled. The signal collecting unit may be configured to repeat collecting a PPG signal if both conditions are not fulfilled.

The processor may be configured to determine whether in a plurality of pairs of pulses in the PPG signal a correlation of a power spectrum density is above a fourth threshold and determining whether a difference in the information about the at least one cardiac cycle is less than a fifth threshold, and the signal collecting unit is configured to repeat collecting a PPG signal if at least one of the conditions is not fulfilled.

The processor may be configured to determine whether in a plurality of pairs of pulses in the PPG signal a correlation of a power spectrum density is above a fourth threshold, whether a difference in the information about the at least one cardiac cycle is less than a fifth threshold, and whether the difference in HR is less than sixth threshold relative to a normal resting heart rate, and the signal collecting unit is configured to repeat collecting a PPG signal using the device if at least one of the three conditions is not fulfilled.

The processor may be configured to determine whether HR is in a normal range for a human and/or animal.

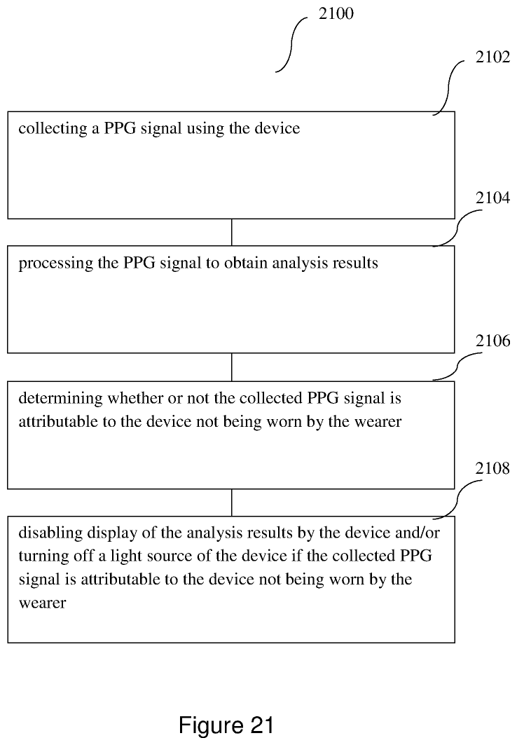

FIG. 21 shows a flowchart 2100 illustrating a method of controlling a wearable device for collecting physiological data of a wearer, according to an example embodiment. At step 2102, a PPG signal is collected using the device. At step 2104, the PPG signal is processed to obtain analysis results. At step 2106, it is determined whether or not the collected PPG signal is attributable to the device not being worn by the wearer. At step 2108, display of the analysis results by the device is disabled and/or turning off a light source of the device if the collected PPG signal is attributable to the device not being worn by the wearer.

Determining whether or not the collected PPG signal is attributable to the device not being worn by the wearer may comprise extracting one or more features from respective time windows of the PPG data.

Determining whether or not the collected PPG signal is attributable to the device not being worn by the wearer may comprise determining a mean and a variance of a DC component of the PPG signal, a variance of an AC component of the PPG signal, a number of times the amplitude of the AC component is saturated, and a number of times the amplitude of the AC component is zero. The method may comprise determining whether the means of the DC component is within a first range, determining whether the variance of the DC component is within a second range, determining whether the variance of the AC component is within a third range, and determining whether a sum of the number of times the amplitude of the AC component is saturated and the number of times the amplitude of the AC component is zero is lower than a first threshold, and determining wrist-off if any one of the conditions is not fulfilled.

Determining whether or not the collected PPG signal is attributable to the device not being worn by the wearer may comprise calculating a signal to noise ratio of the PPG signal.

The signal to noise ratio of the PPG signal may be determined in the frequency domain.

The signal to noise ratio of the PPG signal may be determined based on root mean square of the PPG signal in a signal range and in a noise range, respectively. The method may comprise determining that the device is not worn if the root mean square in the signal range equal to or smaller than a second threshold and/or the root mean square in the noise range is equal to or greater than a third threshold.

The method may comprise determining that the device is not worn if the calculated signal to noise ratio is equal to or smaller than a fourth threshold.

In one embodiment, a wearable device for collecting physiological data of a wearer is provided, the device comprising a signal collection unit for collecting a PPG signal; and a processor coupled to the signal collection unit for processing the PPG signal to obtain analysis results; wherein the processor is configured to determine whether or not the collected PPG signal is attributable to the device not being worn by the wearer and for disabling display of the analysis results and/or turning off a light source of the device if the collected PPG signal is attributable to the device not being worn by the wearer.

The processor may be configured to determine whether or not the collected PPG signal is attributable to the device not being worn by the wearer based on extracting one or more features from respective time windows of the PPG data.

The processor may be configured to determine whether or not the collected PPG signal is attributable to the device not being worn by the wearer based on determining a mean and a variance of a DC component of the PPG signal, a variance of an AC component of the PPG signal, a number of times the amplitude of the AC component is saturated, and a number of times the amplitude of the AC component is zero. The processor may be configured to determine whether the means of the DC component is within a first range, to determine whether the variance of the DC component is within a second range, to determine whether the variance of the AC component is within a third range, and to determine whether a sum of the number of times the amplitude of the AC component is saturated and the number of times the amplitude of the AC component is zero is lower than a first threshold, and to determine wrist-off if any one of the conditions is not fulfilled.

The processor may be configured to determine whether or not the collected PPG signal is attributable to the device not being worn by the wearer based on calculating a signal to noise ratio of the PPG signal.

The signal to noise ratio of the PPG signal may be determined in the frequency domain.

The signal to noise ratio of the PPG signal may be determined based on root mean square of the PPG signal in a signal range and in a noise range, respectively. The processor may be configured to determine that the device is not worn if the root mean square in the signal range equal to or smaller than a second threshold and/or the root mean square in the noise range is equal to or greater than a third threshold.

The processor may be configured to determine that the device is not worn if the calculated signal to noise ratio is equal to or smaller than a fourth threshold.

In one embodiment, a computer-readable medium is provided, comprising instructions which, when executed by a computing device, instruct the device to execute a method according to one of the example embodiments.

The various functions or processes disclosed herein may be described as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the internet and/or other computer networks via one or more data transfer protocols (e.g., HTTP, FTP, SMTP, etc.). When received within a computer system via one or more computer-readable media, such data and/or instruction-based expressions of components and/or processes under the system described may be processed by a processing entity (e.g., one or more processors) within the computer system in conjunction with execution of one or more other computer programs.

Aspects of the systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the system include: microcontrollers with memory (such as electronically erasable programmable read only memory (EEPROM)), embedded microprocessors, firmware, software, etc. Furthermore, aspects of the system may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

The above description of illustrated embodiments of the systems and methods is not intended to be exhaustive or to limit the systems and methods to the precise forms disclosed. While specific embodiments of, and examples for, the systems components and methods are described herein for illustrative purposes, various equivalent modifications are possible within the scope of the systems, components and methods, as those skilled in the relevant art will recognize.

The teachings of the systems and methods provided herein can be applied to other processing systems and methods, not only for the systems and methods described above.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features described for different embodiments, including in the summary section, even if the feature or combination of features is not explicitly specified in the summary section or the detailed description of the present embodiments.

Changes can be made to the systems and methods in light of the above detailed description.

In general, in the following claims, the terms used should not be construed to limit the systems and methods to the specific embodiments disclosed in the specification and the summary, but should be construed to include all processing systems that operate under the summary. Accordingly, the systems and methods are not limited by the disclosure in the example embodiments described herein.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

1. A method of tuning a wearable device for collecting physiological data of a wearer, the method comprising:
collecting a PPG signal using the device;
determining whether or not the collected PPG signal is attributable to the wearer being human or animal; and
discontinuing the tuning if the collected PPG signal is not attributable to the wearer being human or animal;
wherein determining whether or not the collected PPG signal is attributable to the wearer being human or animal comprises determining that the wearer is human or animal if information about at least one cardiac cycle based only on one or more pulses in the collected PPG signal is in a first range.

2. The method of claim 1, wherein the information about the at least one cardiac cycle comprises one or more of a group consisting of a rise time, a fall time, time information relating to the dicrotic notch, time information relating to the diastolic peak, and information about the shape of the at least one pulse in the collected PPG signal or the shape(s) of one or more portions of the at least one pulse in the collected PPG signal.

3. The method of claim 2, comprising, when it is determined that the information about the at least one cardiac is not within a predetermined range, increasing an intensity of a light source for the PPG measurements.

4.-5. (canceled)

6. The method of claim 1, comprising determining whether an amplitude of the collected PPG signal is greater than a first threshold and whether the driving current of the light source is above a minimum, and reducing the intensity of the light source if both conditions are fulfilled.

7.-8. (canceled)

9. The method of claim 1, comprising determining whether an amplitude of the collected PPG signal is equal or greater than a second threshold and whether the driving current of the light source is below the maximum, and increasing the intensity of the light source if both conditions are fulfilled.

10.-12. (canceled)

13. The method of claim 1, comprising determining whether in a plurality of pairs of pulses in the collected PPG signal a correlation of a power spectrum density is above a fourth threshold, whether a difference in the information about the at least one cardiac cycle is less than a fifth threshold, and whether the difference in heart rate, HR, based on the collected PPG signal is less than sixth threshold relative to a normal resting heart rate, and repeating collecting a PPG signal using the device if at least one of the three conditions is not fulfilled.

14. The method of claim 1, comprising determining whether HR is in a normal range for a human and/or animal.

15. A wearable device for collecting physiological data of a wearer, the device comprising:
a PPG sensor;
a signal collecting unit coupled to the PPG sensor for collecting a PPG signal using the PPG sensor for tuning of the device; and
a processor coupled to the signal collecting unit for determining whether or not the collected PPG signal is attributable to the wearer being human or animal;
wherein the processor is configured to discontinue the tuning if the collected PPG signal is not attributable to the wearer being human or animal; and
wherein determining whether or not the collected PPG signal is attributable to the wearer being human or animal comprises determining that the wearer is human or animal if information about at least one cardiac cycle based only on one or more pulses in the collected PPG signal is in a first range.

16. The device of claim 15, wherein the information about the at least one cardiac cycle comprises one or more of a group consisting of a rise time, a fall time, time information relating to the dicrotic notch, time information relating to the diastolic peak, and information about the shape of the at least one pulse in the collected PPG signal or the shape(s) of one or more portions of the at least one pulse in the collected PPG signal.

17. The device of claim 16, wherein the processor is configured, when it is determined that the information about the at least one cardiac cycle is not within the predetermined range, increasing an intensity of a light source of the PPG sensor for the PPG measurements.

18.-19. (canceled)

20. The device of claim 15, wherein the processor is configured to determine whether an amplitude of the collected PPG signal is greater than a first threshold and whether the driving current of the light source is above a minimum, and to reduce the intensity of the light source if both conditions are fulfilled.

21.-22. (canceled)

23. The device of claim 15, wherein the processor is configured to determine whether an amplitude of the collected PPG signal is equal or greater than a second threshold and whether the driving current of the light source is below the maximum, and to increase the intensity of the light source if both conditions are fulfilled.

24.-26. (canceled)

27. The device of claim 15, wherein the processor is configured to determine whether in a plurality of pairs of pulses in the collected PPG signal a correlation of a power spectrum density is above a fourth threshold, whether a difference in the information about the at least one cardiac cycle is less than a fifth threshold, and whether the difference in HR is less than sixth threshold relative to a normal resting heart rate, and the signal collecting unit is configured to repeat collecting a PPG signal using the device if at least one of the three conditions is not fulfilled.

28. (canceled)

29. A method of controlling a wearable device for collecting physiological data of a wearer, the method comprising:
collecting a PPG signal using the device;
processing the PPG signal to obtain analysis results;
determining that the device is not being worn by the wearer if information only about the collected PPG signal does not fulfill at least one condition; and
disabling display of the analysis results by the device and/or turning off a light source of the device if the collected PPG signal is attributable to the device not being worn by the wearer.

30. (canceled)

31. The method of claim 29 or 30, wherein determining whether or not the collected PPG signal is attributable to the device not being worn by the wearer comprises determining a mean and a variance of a DC component of the collected PPG signal, a variance of an AC component of the collected PPG signal, a number of times the amplitude of the AC component is saturated, and a number of times the amplitude of the AC component is zero.

32. (canceled)

33. The method of claim 29, wherein determining whether or not the collected PPG signal is attributable to the device not being worn by the wearer comprises calculating a signal to noise ratio of the collected PPG signal.

34.-37. (canceled)

38. A wearable device for collecting physiological data of a wearer, the device comprising:
a signal collection unit for collecting a PPG signal; and
a processor coupled to the signal collection unit for processing the PPG signal to obtain analysis results;
wherein the processor is configured to determine that the device is not being worn by the wearer if information only about the collected PPG signal does not fulfill at least one condition and for disabling display of the analysis results and/or turning off a light source of the device if the collected PPG signal is attributable to the device not being worn by the wearer.

39. (canceled)

40. The device of claim 38, wherein the processor is configured to determine whether or not the collected PPG signal is attributable to the device not being worn by the wearer based on determining a mean and a variance of a DC component of the collected PPG signal, a variance of an AC component of the collected PPG signal, a number of times the amplitude of the AC component is saturated, and a number of times the amplitude of the AC component is zero.

41. (canceled)

42. The device of claim 38, wherein the processor is configured to determine whether or not the collected PPG signal is attributable to the device not being worn by the wearer based on calculating the signal to noise ratio of the collected PPG signal.

43.-46. (canceled)

47. A computer-readable medium comprising instructions which, when executed by a computing device, instruct the device to execute a method according to claim 1.
